(19) United States
(12) Patent Application Publication
(10) Pub. No.: US 2007/0142727 A1
(43) Pub. Date: Jun. 21, 2007

(54) System and method for analyzing cardiovascular pressure measurements made within a human body

(75) Inventors: Cheng Zhang, Vadnais Heights, MN (US); Thomas W. Piaget, Minneapolis, MN (US); Abhi V. Chavan, Maple Grove, MN (US); Lance E. Juffer, Shoreview, MN (US); Holli Pheil, Blaine, MN (US)

Correspondence Address: FAEGRE & BENSON, LLP;BOSTON SCIENTIFIC PATENT DOCK, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/300,769

(22) Filed: Dec. 15, 2005

(51) Int. Cl. A61B 5/02

(52) U.S. Cl. 600/486

(57) ABSTRACT

A cardiovascular pressure data analyzing system having one or more implanted pressure sensors, an implanted communication device in wireless communication with the sensor and an external data processing unit adapted to use real-time barometric data to calibrate uncalibrated pressure data received from the communication device. The external data processing unit can be portable, is in communication with a remote database to transfer the calibrated pressure data to the remote database, and is capable of providing reprogramming information to the communication device. A method of analyzing pressure data including gathering pressure data from an implanted pressure sensor in a human body, retrieving the pressure data through a communication device implanted in the human body, transmitting pressure data from the communication device to an external data processing unit, and calibrating the pressure data at the external processing unit to compensate for inherent characteristics of the sensor.

### TECHNICAL FIELD

The present invention relates to medical devices implanted within the human body. for measuring blood pressure and other physiologic parameters. More specifically, the invention relates to wireless communication of the physiologic parameter data to an external processing unit.

### BACKGROUND

Medical devices are known that can be implanted within a patient's body for monitoring one or more physiological parameters and/or to provide therapeutic functions. For example, sensors or transducers can be placed in the body for monitoring a variety of properties, such as temperature, blood pressure, strain, fluid flow, posture, respiration, chemical properties, electrical properties, magnetic properties, and the like. In addition, medical devices can be implanted that perform one or more therapeutic functions, such as cardiac pacing, defibrillation, electrical stimulation, and the like.

In many cases, the implanted medical devices are configured or adapted to communicate with external controllers or programmers, for example, to communicate data between the implanted medical device and the external programmers, and/or to activate or otherwise control the implanted medical devices. Typically, implanted medical devices can communicate with the external programmers via a wireless communication link, such as a radio frequency (RF) communication. link, or other acceptable technologies.

As mentioned above, implanted medical devices can be configured to measure or sense a number of different parameters in the body. One parameter of particular interest is blood pressure. The implantable biosensors that measure pressure deep within anatomical structures, however, typically can only communicate the absolute pressure associated with the immediate anatomical environment. These devices are not capable of communicating gauge pressure because they are confined and sealed away from the ambient pressure external to the body.

One way to convert the absolute pressure to a gauge pressure is to compare the absolute measurements taken by the implanted medical device to a measurement of atmospheric pressure taken outside of the body in an external device such as a programmer. U.S. patent application Ser. No. 10/943,626 entitled “SYSTEMS AND METHODS FOR DERIVING RELATIVE PHYSIOLOGIC PARAMETERS,” U.S. patent application Ser. No. 10/943,627 entitled “SYSTEMS AND METHODS FOR DERIVING RELATIVE PHYSIOLOGIC PARAMETERS USING AN EXTERNAL COMPUTING DEVICE,” U.S. patent application Ser. No. 10/943,269 entitled “SYSTEMS AND METHODS FOR DERIVING RELATIVE PHYSIOLOGIC PARAMETERS USING A BACKEND COMPUTING SYSTEM,” and U.S. patent application Ser. No. 10/943,271 entitled “SYSTEMS AND METHODS FOR DERIVING RELATIVE PHYSIOLOGIC MEASUREMENTS USING AN IMPLANTED SENSOR DEVICE” each disclose methods and apparatuses for converting the absolute pressure readings of the implanted medical device to a gauge reading in an external device and are hereby incorporated by reference in their entirety for all purposes.

However, sensors of the type implanted to read, for example, blood pressure within the human body provide raw data readings that may vary from the actual absolute pressure because individual implanted medical devices may have differing linearity, gain, and offset factors from the ideal values and from one sensor to the next, thereby introducing error into the pressure data. To compensate for these potential errors, sensors need to be calibrated. It is known to provide calibration of sensor information in the implanted medical device. Using the implanted medical device for calibration purposes presents other challenges, most notably the challenge of providing adequate computing power within the implanted medical device to obtain the desired precision and accuracy, and providing added electrical power required to make such calculations over time.

Thus, a need exists for systems, methods, and/or devices for providing calibration of measured physiologic parameters, such as pressure, temperature and others, based on ambient or other environmental conditions and the inherent error introduced by individual sensors without increasing the power consumption of implanted medical devices.

### SUMMARY

The invention is directed toward a system for analyzing cardiovascular pressure data within a human body. The system includes a sensor implanted in the human body for measuring pressure data, an implanted communication device that can communicate wirelessly with the sensor to receive pressure data from the sensor in an uncalibrated form, and an external data processing unit capable of receiving the uncalibrated pressure data from the communication device through wireless communication and calibrating the pressure data. The external processing data unit includes a barometric pressure sensor to provide real-time barometric data used by the external processing data unit to generate relative pressure data.

The invention is also directed toward a system for analyzing blood pressure data corresponding to blood pressure in a human body including implanted sensor means for collecting diagnostic information within the human body. The system further includes communication means for transmitting the diagnostic information and external data processing means for automatically receiving and calibrating the diagnostic information.

The invention is further directed toward a method of analyzing pressure data in a human body. The method includes using an implanted pressure sensor to gather pressure data from within the body. Once the pressure is gathered by the sensor it is transmitted to an implanted communication device and subsequently transmitted to an external data processing unit, which calibrates the pressure data, by compensating for inherent characteristics of the sensor.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a system for using implanted medical devices for measuring physiologic parameters within a human patient, transferring measured data outside of the human patient with or without human intervention, and calibrating the measured data external to the human patient according to one exemplary embodiment of the invention.

FIG. 2 is a block diagram of a communication device implanted within a human patient as part of the system illustrated in FIG. 1.

FIG. 3 is a block diagram of a physiologic sensor implanted within a human patient and adapted to communicate with the communication device of FIG. 2.

FIG. 4 is a block diagram of an external processing device adapted to communicate with one or more implanted medical devices with the human patient of FIG. 1.

FIG. 5 is a functional block diagram of a process of measuring and storing physiologic data within the human patient of FIG. 1.

FIG. 6 is a functional block diagram of a process of transferring the physiological data measurements illustrated in FIG. 6 out of the human patient for calibration and analysis.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

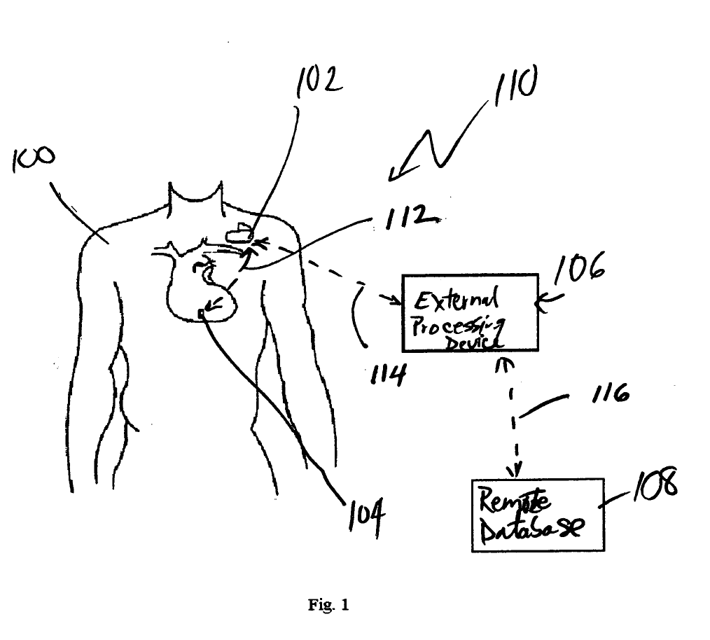

FIG. 1 illustrates a system 110 for measuring physiologic parameter data in a human patient 100 and precisely calibrating the measured data in accordance with one exemplary embodiment of the invention. System 110 includes at least one communication device 102 and at least one physiologic sensor 104 implanted in the human patient 100, an external processing device 106, and a remote database 108. Communication links 112, 114, and 116 provide the ability to transfer information between the various components of system 110.

Communication device 102 can be any type of implantable medical device capable of communicating wirelessly with the external processing device 106 to transmit sensor data collected within the human patient 100 to the external processing device. For example, the communication device 102 can be a pulse generator that is configured to obtain measurements of physiologic parameters from the body of a human patient and provide therapy to the patient. Examples of the type of pulse generators that can include communication device 102 are a pacemaker, an implantable cardioverter defibrillator, a cardiac resynchronization device, a bi-ventricular pacer, a ventricular assist blood pump, a drug delivery pump, a drug infusion device, and a neurostimulating device. This list is intended to be non-limiting, as other acceptable pulse generators can be used. In one exemplary embodiment, communication device 102 is a pacemaker or implantable cardioverter defibrillator. Alternatively, the communication device 102 need not also provide a therapeutic function. For example, communication device 102 can be a device dedicated solely to communication with other devices located within and/or external to the body of the human patient 100. Other acceptable communication devices may perform other non-therapeutic functions. As an example, communication device 102 can measure physiologic parameter data.

Physiologic sensor 104, in the illustrated embodiment, is a pressure sensor for measuring the absolute blood pressure of the human patient 100. Physiologic sensor 104 can be located in various locations within the human patient 100, including within the heart 120 or any one of a number of blood vessels within the human patient (not shown). In addition, the system 110 can include a plurality of physiologic sensors 104 to measure other parameters such as body temperature, oxygen or glucose levels in blood, blood flow, respiration, and posture. Alternatively, a single physiologic sensor 104 can measure a plurality of parameters. Alternatively still, one or more physiologic sensor elements similar to those in physiologic sensor 104 (described below) may be located within the communication device 102. Some of these parameters can be useful when analyzing blood pressure data. Others can be useful in other physiologic analyses. While one exemplary embodiment described here relates to a system for measuring and calibrating blood pressure data that provides analysis of the cardiovascular system of the human body, alternative embodiments can measure and calibrate data related to other body functions. In addition, while the exemplary embodiment of system 110 describes a physiologic sensor 104 separate from the communications device, in an alternate embodiment the system can include a communications device 102 with one or more sensor elements integrated therein. Such a system may or may not have a physiologic sensor 104 mounted in a discrete package from the communications device 102.

The external processing device 106 is located external to the human patient 100. In one exemplary embodiment, the external processing device 106 is a hand held device. Alternatively, the external processing device 106 can be a portable device of any size. Alternatively, still, external processing device 106 is a stationary device. In other embodiments, external processing device 106 can be a commercial electronic device such as a pager, watch, cell phone, or personal data assistant (PDA) adapted to perform the functions of the external processing device as required in system 110.

External processing device 106 is capable of communicating with implantable medical devices such as communication device 102 to receive physiologic data measured within the human patient. In addition, the external processing device 106 performs mathematical computations to calibrate the received physiologic data. Further, the external processing device 106 is capable of transmitting information to the implantable medical devices such as communication device 102 to initiate data transfer from the communication device to the external processing device and for other purposes that will become apparent.

The remote database 108, in the illustrated embodiment, can be located anywhere relative to other components of the system 110. The remote database 108 can include information transmitted from the external device 106 via communication link 116. In addition, the remote database 108 may include information that can be used by the external processing device 106. For example, it may have calibration data for each unique physiologic sensor that the external processing device can use in its calibration procedures. Further, the remote database 108 can include information and code for any software or calibration modifications that are available for any implanted medical devices or other data. The remote database is capable of storing all relevant diagnostic information obtained from the human patient 100 as well as information from other human patients. The remote database can provide a variety of data analyses, including trend analysis of data collected from the human patient 100 over time. The remote database 108 provides access to this information for those individuals who are properly and legally authorized to obtain it and who require that information to provide health care for the human patient.

In one exemplary embodiment, communication link 112 is a communication link between various implanted medical devices of system 110. For example, the communication device 102 communicates with the physiologic sensor 104 located external to communication device 102 via communication link 112. The nature of the information transferred between communication device 102 and the physiological sensor 104 will be discussed below. Communication link 114 is a communication link that enables communication between one or more of the implanted medical devices and the external computing device 106. For example, communication device 102 can communicate with the external computing device 106 via communication link 114 to export information collected within the human body 100 to the external computing device. Communication link 114 also allows external computing device 106 to communicate information to implanted medical devices such as the communication device 102. The nature of the information passed from the external communication device 106 and implanted medical devices will be discussed in more detail below. Communication link 116 provides a communication path to allow for information exchange between the external processing device 106 and remote database 108 as will be discussed below.

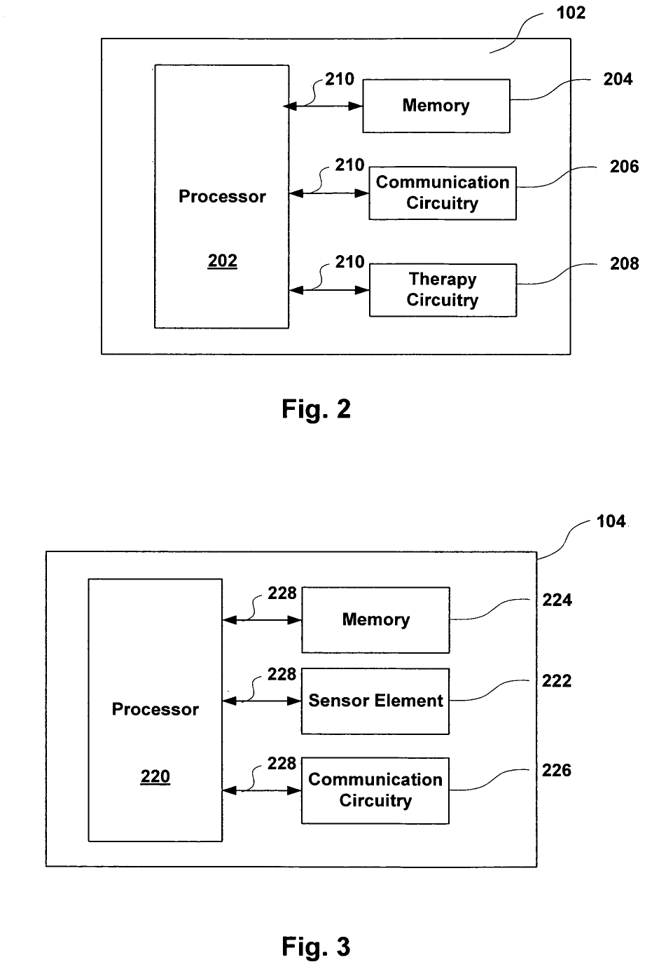

FIG. 2 is a block diagram of the communication device 102 according to one exemplary embodiment illustrating some of the more pertinent aspects of the communication device circuitry. Communications device 102 includes a processor 202, memory 204, communication circuitry 206, and therapy circuitry 208. Memory 204, communication circuitry 206, and therapy circuitry 208 are in electrical communication with processor 202, as is represented by circuit connections 210. Alternatively, as described above communication device 102 can include one or more sensor circuitry (not shown) of the type described below in conjunction with physiological sensor 104.

Processor 202 can be any suitable processing device, such as a microcontroller or any circuitry that provides processing capability to control communication, memory storage and therapy functions performed by the communication device 102. Memory device 204 can be an EEPROM or any other suitable memory device. Communication circuitry 206 includes necessary circuitry for a wireless connection such as, for example, a near field and/or far field radio frequency (RF) communication connection, an acoustic communication connection (e.g., an ultrasound connection), Bluetooth, or any other suitable wireless communication connection. It should be appreciated that these examples are for illustrative purposes only. Any other suitable communication can be used and thus the supporting circuitry would be included in the communication device 102. Alternatively or in addition, the communication circuitry 206 can include drivers for a wired connection between the communication device 102 and the physiologic sensor 104.

In one exemplary embodiment, the communication device 102 provides a gateway between the physiologic sensor 104 and the external processing device 106. Data collected by the physiologic sensor 104 is communicated to and stored in the communication device 102, which then communicates the data to the external processing device 106 at the appropriate time. Thus, communication circuitry 206 can include more than one type of communication circuitry. For example, the communication circuitry 206 may have RF circuitry for communication link 114 between the communication device 102 and the external processing device 106 and an acoustic communication connection for communication link 112 between the communication device and the physiologic sensor 104. Alternatively, system 110 can include a plurality of physiologic sensors 104 having a plurality of communication methods, thereby requiring that communication link 112 include circuitry for each required communication method.

Therapy circuitry 208 includes circuitry for providing one or more therapeutic functions to a patient including, for example, appropriate circuitry for providing heart pacing therapy, cardiac defibrillation therapy, cardiac resynchronization therapy, or any other therapy associated with a suitable communication device 102. Alternatively, as described above, communication device 102 does not include a therapeutic function and therefore does not include any therapy circuitry.

Physical implementations of communication device 102 may vary widely. For example, an integrated circuit, such as an application specific integrated circuit (ASIC), that includes the functions represented by processor 202 may also include memory device 204 and some or all of the circuitry necessary for other functions, including communication circuitry 206 and therapy circuitry 208. Further, communication device 102 can include other circuitry not represented in FIG. 2, such as power-saving circuitry, random access memory and a variety of other circuits necessary for the function of the communication device. The descriptions here are for illustrative purposes only and are not intended to be limiting.

FIG. 3 is a block diagram of the physiologic sensor 104 according to one exemplary embodiment. Physiologic sensor 104 includes a processor 220, one or more sensor elements 222, memory 224, and communication circuitry 226. Sensor element 222, memory 224, and communication circuitry 226 are in electrical communication with processor 220, as is represented by circuit connections 228. Like processor 202, processor 220 can be any suitable processing device or circuitry.

Sensor element 222 includes a sensing component exposed to a given physiologic phenomena, such as blood pressure, to provide an electrical signal that characterizes the relative amount of the phenomena present. Sensor element 222 further includes any circuitry required to condition and/or amplify a signal from the sensing component. For example, a sensor element 222 that senses blood pressure would include a pressure sensing component and associated signal conditioning circuitry. The signal conditioning circuitry conditions the signal to prevent saturation of the sensing component, to amplify the signal provided by the pressure sensing component and/or to convert an analog signal into a digital signal. The conditioned signal of sensor element 222 provides an analog or digital output signal having a range of values corresponding to the range of pressure capable of being measured by the sensor element. It is to be understood that although the sensor element 222 provides a conditioned signal, that signal is not calibrated.

Memory device 224 includes sufficient memory space for any software, firmware, or other code related to the operation of physiologic sensor 104, including all software to perform communication functions. In addition, memory device 224 can hold parameters such as calibration data and sensor identification information unique to a given physiologic sensor. As described above, the sensor element 222 provides an output signal that is a function of pressure. While individual sensor elements 222 may have similar relationships between pressure and their respective output values, there may be somewhat significant variations between the output signal of individual sensors as a function of pressure. To compensate for the variations, calibration data is used to describe the relationship for each sensor with suitable precision. The calibration data can include factors for linearity, gain and offset, for example. Such data is loaded into memory during a manufacturing testing process.

In one exemplary embodiment, physiologic sensor 104 is capable of being reprogrammed after implantation into the human patient 100. Reprogramming can include reprogramming operational code, calibration data, or other software, firmware, or code within the sensor. Thus, at least a portion of memory device 224 requires reprogrammable memory such as an electrically erasable programmable read only memory (EEPROM), reprogrammable flash memory, or other acceptable reprogrammable memory.

In one exemplary embodiment, communication circuitry 226 includes necessary circuitry for wireless connection with other implantable medical devices such as communication device 102 or other physiologic sensors 104 on communication link 112. Alternatively, communication circuitry 226 can also include circuitry to communicate with the remote processing device 106. For example, to provide capability for the physiologic sensor 104 to communicate via communication link 112 and/or communication link 114, communication circuitry 226 can include circuitry for a near field and/or far field radio frequency (RF) communication link, an acoustic (e.g., ultrasound) communication link, Bluetooth, or any other suitable wireless link connection or combination of links. Alternatively, the communication circuitry 226 can include drivers for a wired connection between the physiologic sensor 104 and the communication device 102.

Physical implementations of physiologic sensor 104 may vary widely, including, without limitation, an integrated circuit such as an ASIC that performs some or all the functions represented by processor 220, memory device 224 and the circuitry necessary for other functions, including communication circuitry 226 and sensor element 222. Further, physiologic sensor 104 can include other circuitry not represented in FIG. 3, such as power-saving circuitry, random access memory and a variety of other circuits necessary for the function of the physiologic sensor.

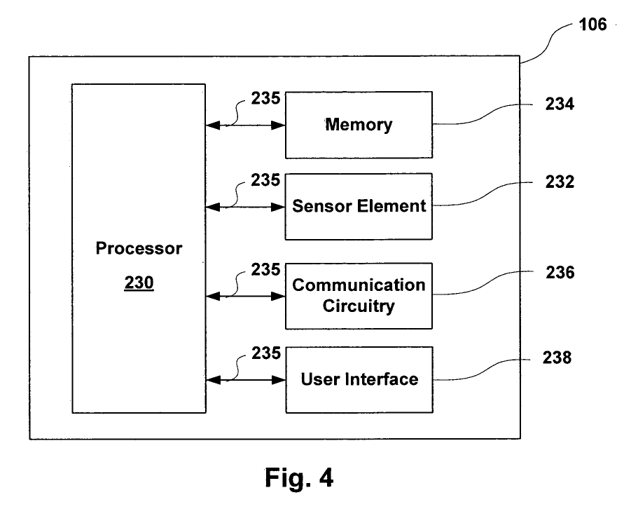

FIG. 4 is a block diagram of the external processing device 106 according to one exemplary embodiment. The external processing device 106 includes a processor 230, a sensor element 232 for sensing ambient air pressure, memory 234, communication circuitry 236 and a user interface 238. Sensor element 232, memory 234, and communication circuitry 236 are in electrical communication with processor 230, as is represented by circuit connections 235.

Processor 230 can be any suitable processing device or circuitry for performing the functions required by external processing device 106. Communication circuitry 236 includes necessary circuitry to support wireless connection with implantable medical devices such as communication device 102 or physiologic sensor 104 via communication link 114. Communication link 114 may utilize one of the following communication techniques and require the necessary circuitry: a near field and/or far field radio frequency (RF) or an acoustic communication (e.g., ultrasound) technique. These examples are for illustrative purposes only. Any other suitable communication can be used and in such a case the supporting circuitry would be included in the external processing device 106.

Sensor element 232 includes a sensing component to sense the atmospheric pressure and any circuitry required to condition and/or amplify a signal from the sensing component. Sensor element 232, in one exemplary embodiment, is located within the external processing device 106. Sensor element 232 is electrically coupled to the processor 230 through circuit connections 235 to provide real time atmospheric readings. Alternatively, external processing device 106 may not have an integral sensor element 232, but rather can be in communication with an external atmospheric sensor (not shown) to receive real time atmospheric readings.

Memory 234 includes an EEPROM, flash memory, a hard disk drive, or any other suitable memory device with sufficient memory space for any software, firmware or other code related to the operation of the external processing device 106. In addition, memory device 224 can hold parameters such as calibration data and sensor identification information unique to particular physiologic sensors. Further, memory 234 has sufficient size to store information transmitted from implanted medical devices as well as information such as reprogramming data for implanted medical devices, if necessary.

External processing device 106 can also include, in one embodiment, a user interface 238. User interface 238 can include a display screen to display information to the human patient, health care workers, or others. In addition, external processing device 106 can include a keyboard, keypad, input switches, or other suitable devices for inputting information into the external device.

External processing device 106 is capable of communicating with the remote database 108 over a communication link 116 to exchange information between the external computing device and the remote database. The communication link 116 between the external computing device 106 and the remote database 108 can include a direct telecommunication link between the external device and the remote database utilizing modems, an Internet based link, near field and/or far field RF communication connection, Bluetooth, or any other similar data communication format. The nature of the information exchanged between the external computing device and the remote database will be discussed below.

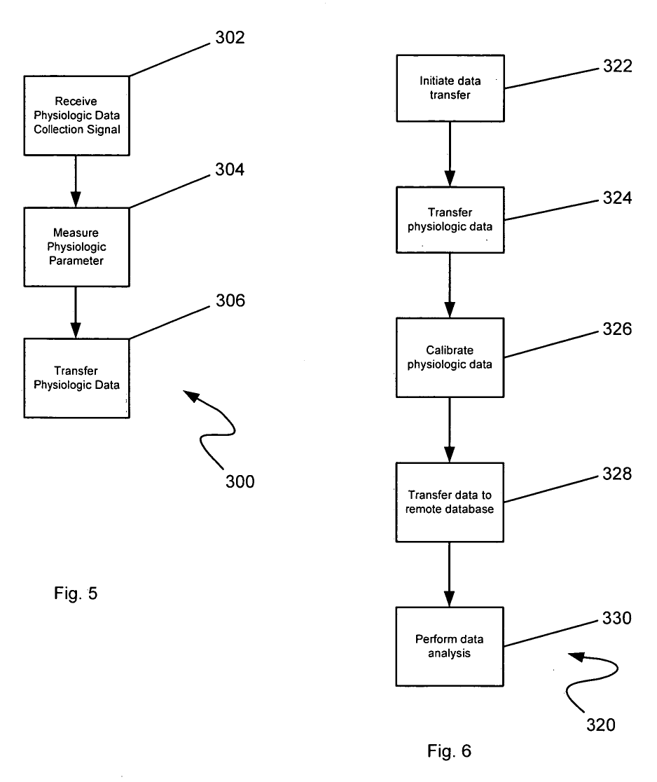

FIG. 5 illustrates a functional flow diagram of a sensor data collection event 300 according to one exemplary embodiment. Data collection event 300 includes collecting sensor data by a physiologic sensor 104 and storing that information within the human patient 100. While the process described below is related to a single physiologic sensor 104 reading a single parameter, it will be appreciated, as described above, that some human patients may have a plurality of physiologic sensors. Further, one or more physiologic sensors 104 in the human patient 100 may measure more than one parameter. In such cases, the sensor data collection event 300 can be performed to collect data from each of the plurality of parameters, including some instances where a single sensor provides data for a plurality of sensors. Further, it should be appreciated that when a human patient 100 has a plurality of implanted physiologic sensors 104, each of the plurality of physiologic sensors can collect data simultaneously.

Step 302 initiates the physiologic sensor data collection event 300. In the one embodiment, the physiologic sensor 104 functions in a reduced function or “sleep” mode when not actively reading sensor data. The sleep mode allows the physiologic sensor 104 to reduce the power consumption of its circuitry and thereby improve battery life. Periodically, the communication device 102 initiates a communication via communication link 114 with the physiologic sensor 104 to arouse the physiologic sensor from sleep mode to take a sensor reading. Alternatively or in addition, and as described above, communication device 102 can include a sensor device. Thus, the communication device 102 can initiate its own data collection periodically along with communicating via communication link 114 with any physiologic sensor 104 that may be implanted in the human patient 100.

The frequency of the periodic data collection events are preset or preprogrammed into the communication device 102. For example, the communication device 102 may be programmed to initiate the sensor data collection event 300 four, six, or eight times per day, although the frequency may vary significantly without departing from the scope of the invention. Further, the frequency may be altered by reprogramming the communication device 102 after it has been implanted within the human patient. Alternatively or in addition, the communication device 102 may initiate the sensor data collection event 300 in response to a communication request received from an external source. For example, the external processing device 106 may initiate a request to the communication device 102 to initiate the sensor data collection event 300. In yet another embodiment, the external processing device 106 may initiate the sensor data collection event 300 with the physiologic sensor 104 directly. For example, the human patient 100 or health care provider may manipulate the external processing device 106 to initiate the data collection signal. In still another embodiment, the physiologic sensor 104 may periodically initiate the sensor data collection event 300 in response to a periodic signal generated within the physiologic sensor itself.

When the physiologic sensor 104 receives a communication from another device such as the communication device 102 to initiate the sensor data collection event 300, the physiologic sensor verifies that it has received a signal from a device properly initialized to receive sensor data. For example, the communication device 102 can include a unique sensor identification in its original transmission to the physiologic sensor 104 to provide verification. Alternately, the physiologic sensor 104 can request the sensor identification information from the communication device 102, at which point the communication device 102 would provide sensor identification information. In one exemplary embodiment, the sensor identification information is encrypted for data security purposes. If the physiologic sensor 104 does not determine that the request for sensor data that it received was intended to prompt the physiologic sensor to perform the data collection event 300, the physiologic sensor does not perform the sensor data collection event. Alternatively still, the physiologic sensor 104 will respond to a request from a device such as communication device 102 without requiring verification.

As described above, it should be appreciated that a single human patient can have a plurality of physiologic sensors 104 located within the human body. A single communication device 102 can send a communication to a plurality of physiologic sensors 104 to initiate the sensor data collection event 300. Alternatively, the human patient can also have a plurality of implanted communication devices 102 that can initiate a sensor data collection event 300 with one or more physiologic sensors 104.

Once the physiologic sensor 104 establishes that the request for data collection is proper or has “wakened” from sleep mode, the physiologic sensor performs the step 304 of measuring the physiologic parameter. In one exemplary embodiment, the physiologic sensor 104 takes one or more readings of the relevant physiologic parameter over a given period of time, for example, for ten to fifteen seconds. Alternatively, the physiologic sensor 104 can measure the physiologic data for any length of time and take any number of readings over that length of time. Once the uncalibrated physiologic data is collected by the sensor element 222, it is stored in memory 224. The physiologic sensor 104 may store a single data point measured by the sensor element 222, an average of several data points taken over a given time interval and/or a plurality of data points measured by the sensor over a given amount of time.

When the physiologic parameter has been measured, step 306 is performed to transfer the uncalibrated physiologic data. In one exemplary embodiment, the physiologic data is transferred from the physiologic sensor 104 to the communication device 102 and stored in memory 204. The transferred data can be given a time stamp in memory 204 to correlate the data with the time that it was actually measured. Alternatively, the transferred data has a time stamp provided when the data was collected in the physiologic sensor 104. Alternatively still, no time stamp is provided by the physiologic sensor 104 or the communication device 102.

In one exemplary embodiment, the physiologic data is communicated via the communication link 112 between the physiologic sensor 104 and the communication device 102 and the data is then stored in the memory 204 of the communication device. Once the data collection event 300 is completed, the physiologic sensor 104 returns to sleep mode. Alternatively or in addition, the communication device 102 includes a sensor and the physiologic data is transferred internally within the communication device and stored in memory 204.

FIG. 6 illustrates a function flow diagram of analysis 320 of the process of collecting, transferring, and analyzing physiologic data according to one exemplary embodiment. Step 322 is the step of initiating data transfer of physiological data collected from an implanted medical device within the human patient 100 to the external processing device 106. In one exemplary embodiment, step 322 includes a data transfer request in the form of a communication sent via communication link 114 between the external processing device 106 and an implanted medical device such as communication device 102. In one exemplary embodiment, the communication device 102 is designated to initiate the data transfer request of step 322 by sending a data transfer request to the external processing device 106. Alternatively, the external processing device 106 can be so designated. The automated data transfer request can be sent periodically, such as once hourly, daily, weekly, or upon any other periodic time frame. Alternatively still, the external processing device 106 can be manipulated to request a data transfer at any time.

Once the data transfer request communication has been sent by, for example, the communication device 102, the communication device awaits a response from the external processing device 106 to establish communication. The external processing device 106 may not respond, for example, if the human patient 100 is out of the communication range of the external processing device. If the external processing device 106 does not make communication with the communication device 102, it can make subsequent attempts, either shortly after the failed attempt, or at the next appointed time. If the communication device 102 is designated as the device to initiate step 322 and the external processing unit 106 has not received a request for physiological data transfer for a given period of time, the external processing unit can periodically attempt to establish communication with the communication device to initiate a data transfer. If the external processing unit 106 is designated as the device to initiate step 322 and the communication device 102 has not has not received a request for a given period of time, the communication device can likewise periodically attempt to initiate a transfer.

Once communication has been established between the external processing device 106 and the communication device 102, the communication device must determine whether the external processing device 106 has authorization to receive data from the communication device 102. Communications device 102 will request, or external processing device 106 will provide, as part of its initial message, information identifying the unique communication device 102 and/or the physiologic sensor(s) 104 located within the human patient. If the external processing device 106 provides the proper information, the step 322 of initiating data transfer will have been successfully completed.

It should be understood that a plurality of external processing devices 106 may have the proper sensor or communication device identification information for a given human patient. Therefore, it is possible that a plurality of external processing devices 106 may be in range of the human patient and therefore be able to successfully complete step 322 simultaneously. In one embodiment, each of the external processing devices that have successfully completed step 322 can simultaneously move to the subsequent step 324 of receiving information from the communication device 102 and the communication device can effectively broadcast to all of the authorized external processing devices 106.

Alternatively, the plurality of external processing devices 106 may have a given priority status, and the communication device 102 will establish communication with the external processing device with the highest priority. For example, a stationary external processing device 106, which may be located in a medical center, may have a higher priority than a handheld external processing device. In such a situation, the communication device will communicate only with the external processing device 106 having the highest priority. Various communication protocols may be used to restrict communication from the communication device 102 to only the highest priority external processing device 106.

The step 324 of transferring physiologic data from the human patient 100 to the external processing device 106 includes the transfer of physiologic data from the communication device 102 to the external processing device. In one embodiment, communication device 102 provides data previously collected from sensors such as the physiologic sensor 104 and time stamp information. Alternatively, the communication device 102 initiates data collection event 300 once the step 322 step of initiating data transfer has been successfully completed. In that case, the step 324 of transferring data would not require transferring time stamp information.

In addition, the step 324 of transferring of physiologic data can include transferring calibration data unique to the physiologic sensor 104. Alternatively, calibration data may be previously loaded within the external processing device 106, and therefore that information would not need to be transmitted from the communication device 102 to the external processing device. Alternatively still, the communication device 102 can transmit a calibration version identifier to indicate which version of calibration data is stored within the physiologic sensor 104. When the calibration version identifier in the physiologic sensor 104 matches that of the calibration version identifier in the external processing device 106, no need exists to download calibration data with each transmission. If however, either the communication device 102 or the external processing device 106 has a newer version of calibration data for the particular physiologic sensor 104 in question, the device with the newer calibration version will send that information to the other device so that the other device can reprogram its memory with the more current information.

The step 324 of transferring physiologic data from the human patient to the external processing device 106 may include transferring the data collected and stored during a single sensor data collection event 300, whether that is a single data point from a single physiologic sensor 104 or a plurality of data points. Alternatively, the step 324 of transferring physiologic data can include transferring data from a plurality of previously conducted data collection events 300. It should be appreciated that the step 324 of transferring physiologic data can include the transfer of data from a plurality of physiologic sensors 104 through one or more communication device 102 devices.

Each of the transfers requires that the external device 106 identify each of the sensors from which data is to be transferred. All of the transferred information is stored in the memory 234 of the external processing device 106. In one exemplary embodiment, the external device 106 provides a time stamp for the data when it is transferred from the human patient, if the transferred data does not include a time stamp, for example, if step 300 is performed in response to a successful completion of step 322 as described above. In addition, the external processing device 106 reads the atmospheric pressure from the sensor located within the external processing device 106 or, alternatively, receives atmospheric pressure from an externally located sensor. The atmospheric information is stored within the memory 234 of the external processing device with a time stamp, if collected previously or with the collected data if collected simultaneously with the data collection event 300.

It should be appreciated that just as multiple external processing devices 106 can be properly initialized to communicate with a single communication device 102 or plurality of communication devices within a single patient to receive physiologic data measured by one or more physiologic sensors 104 within the human patient 100, a single external processing device 106 can be properly initialized to communicate with a plurality of different communication devices 102 located within a plurality of human patients. The data associated with each of the human patients collected within the external processing device 106 is stored in memory 234 in such as way as to keep the data from one human patient distinct from the data collected from others.

Once data has been transferred from the human patient to the external processing device 106, the external processing device performs the step 326 of calibrating the physiologic data. The step 326. of calibrating the physiologic data includes converting raw data into more accurate blood pressure data, which includes several different processes. One step in the calibration process includes computing the effects of the individual sensor error. Calibration data, stored in the human patient (in physiologic sensor(s) 104 and/or communication device(s) 102) and in the external processing device 106 calibrate the data to account for the known variation in the given sensor's linearity, gain, and offset. Another step in the calibration process is to subtract atmospheric pressure from the readings taken in the human patient 100. The atmospheric pressure readings are taken in real-time by the sensor 232 in the external processing device 106 or another atmospheric sensor, which has its data transferred to the external computing device 106.

In addition, other physiologic data transferred from the human patient, including posture, body temperature data, and pressure data taken from multiple locations, which can be used to calibrate blood pressure data. Further still, step 326 can include techniques to compensate for the effects of respiration on blood pressure data. These are examples of the type of data that can be used to provide accurate and precise blood pressure data. External processing device 106, not being bound by the power, computational limitations, or other restrictions inherent in implantable medical devices such as communication device 102 are able to more accurately calculate physiological phenomena such as blood pressure. In one embodiment, the external processing device performs calculations using third order polynomials to describe the relationship between stored physiological data and the actual physical phenomena.

While the process above is used to describe the analysis of blood pressure data, any number of physical parameters of the human body can be similarly analyzed. For example, similar devices and processes can be used to analyze blood oxygen or glucose levels, blood flow, and the presence of chemicals in the body.

The result of the data calculation can be displayed for review by the external processing device 106. As described above, the external processing device may have a user interface 238, including a display screen to display information to the human patient, health care workers, or others. In addition, external processing device 106 can include a keyboard, input switches or other suitable devices for inputting information into the external processing device, for example to request that the device display certain information on its display.

Once the step 326 of calibrating physiologic data has been completed, the step 328 of transferring calibrated data to remote database 108 via communication link 116 is performed. Data transferred to the remote database 108 can be stored with any other information previously collected from the human patient 100. As a result, the step of data analysis 330 can be performed on the data received. For example, trend analysis, which may provide information about chronic conditions, potential health concerns either previously known or unknown or any other relevant information. Further, once the information is stored in the remote database, those with proper authorization will be able to access all relevant health information. In one embodiment, the external processing device 106 can be used to access health data from the remote database 108.

In addition to initiating the transfer of information from the human patient, the external processing device 106 can interact with the implanted medical devices such as the communication device 102 to perform other functions. For example, the external processing device 106 can initiate a reprogramming process in the communication device 102. Any of the software code within the communication device 102 can, in one embodiment, be reprogrammed. This can include reprogramming the sensor identification information, if necessary, of the other sensors located within the human patient, adding functional features or modifying existing features. By communicating via communication link 114, the external processing device 106 is capable of initiating and providing the relevant information to complete the initialization.

Further, the external processing device 106 may have the capability of communicating directly with a physiologic sensor 104 to provide reprogramming tasks, such as, for example adjusting calibration data or any other programming tasks. Alternatively, the external processing device may be able to communicate with the communication device 102 and direct the communication device to communicate with the physiologic sensor 104 via communication link 114 and use the communication device as a gateway for the purposes of reprogramming the physiologic sensor 104.

The present invention provides a number of advantages. Calibrating data in the external processing device reduces the electrical power used by implanted medical devices. In addition, the computing power available in the external processing device allows for more precise calibrations than would be found in an implanted medical device. Further, by using a variety of different sensors located within the human patient, the present invention can provide more meaningful data. For example, as posture and body temperature can impact blood pressure readings, information from sensors to sense those physiologic parameters and provide precise calibration of their readings further improves the data available to health care professionals.

In addition, the external processing device automatically initiates the data collection and calibration, thereby improving the data collection process by allowing ongoing periodic data collection without human intervention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system for analyzing cardiovascular pressure data within a human body, comprising:
a sensor implanted at a location in the human body for measuring pressure data;
a communication device implanted in the human body and in wireless communication with the sensor for receiving the pressure data from the sensor in an uncalibrated form; and
an external data processing unit having a barometric pressure sensor and capable of wireless communication with the communication device for receiving the uncalibrated pressure data,
wherein the external data processing unit is adapted to use real-time barometric data to calibrate the uncalibrated pressure data, and generate relative pressure data in real-time based on barometric and calibrated pressure data.

2. The system of claim 1, wherein the communication device is a pulse generator.

3. The system of claim 1, wherein the communication device is associated with a unique identification parameter for enabling wireless communication of the uncalibrated pressure data from the communication device to the external data processing unit.

4. The system of claim 3, wherein the external data processing unit includes a plurality of unique identification parameters, each unique identification parameter uniquely identifying each of a plurality of communication devices, and enabling wireless communication of pressure data to the external data processing unit from the plurality of communication devices.

5. The system of claim 1, wherein the sensor is associated with a unique identifier, whereby the external data processing unit can uniquely identify the sensor to calibrate the uncalibrated pressure data based on calibration characteristics associated with the sensor.

6. The system of claim 5, wherein the external data processing unit is in communication with a network database having stored therein calibration characteristics corresponding to the unique identifier, and wherein the external data processing unit accesses the calibration characteristics from the network database.

7. The system of claim 1, wherein the external data processing unit further includes a capability for providing reprogramming information to the communication device.

8. The system of claim 1, wherein the external data processing unit is portable.

9. The system of claim 1, wherein the sensor includes calibration information for adjusting pressure data collected by the sensor and wherein the calibration information is accessible by the external data processing unit.

10. The system of claim 1, wherein the sensor is a first sensor and the location is a first location, the system further comprising a second sensor implanted at a second location in the human body for measuring data related to a physiologic parameter within the human body, the second sensor in wireless communication with the communication device, and wherein the external data processing unit is capable of wireless communication with the communication device for receiving data measured by the second sensor.

11. The system of claim 10, wherein the physiologic parameter is related to pressure at the second location.

12. The system of claim 10, wherein the physiologic parameter is related to the posture of the human body at the second location.

13. The system of claim 10, wherein the physiologic parameter is related to body temperature at the second location.

14. The system of claim 10, wherein the physiologic parameter is related to respiration.

15. The system of claim 10, wherein the physiologic parameter is related to one of blood oxygen level, blood glucose level, and blood flow at the second location.

16. The system of claim 1, further comprising a remote database in communication with the external data processing unit, wherein the external data processing unit is adapted to transfer the calibrated pressure data to the remote database.

17. A method of analyzing pressure data, comprising:
gathering pressure data at a pressure sensor implanted in a human body;
retrieving the pressure data from the pressure sensor by a communication device implanted in the human body;
transmitting pressure data from the communication device to an external data processing unit; and
calibrating the pressure data at the external processing unit to compensate for inherent characteristics of the sensor.

18. The method of claim 17, further comprising a step of establishing communication between the communication device and the external processing unit prior to the step of transmitting the pressure data from the communication device to the external unit.

19. The method of claim 18, wherein the step of establishing communication between the communication device and the external processing unit is initiated by the communication device.

20. The method of claim 18, wherein the step of establishing communication between the communication device and the external processing unit is automatically initiated in response to a predetermined event.

21. The method of claim 18, wherein the step of establishing communication between the communication device and the external data processing unit includes providing proper identification information from the external data processing unit to the communication device.

22. The method of claim 21, wherein a plurality of external data processing units simultaneously attempt to establish communication with the communication device and further comprising the step of selecting one external data processing unit for communication with the communication device.

23. The method of claim 17, further comprising the step of calibrating the pressure data to compensate for atmospheric pressure.

24. The method of claim 17, further comprising the step of calibrating the pressure data to compensate for other physiologic parameters.

25. The method of claim 24, wherein the step of compensating for other physiologic parameters includes compensating for respiration by the human body.

26. The method of claim 24, wherein the step of compensating for other physiologic parameters includes compensating for posture of the human body.

27. The method of claim 24, wherein the step of compensating for other physiologic parameters includes compensating for temperature of the human body.

28. A system for analyzing blood pressure data corresponding to blood pressure in a human body, the system comprising:
implanted sensor means implanted for collecting diagnostic information within the human body;
implanted communication means for collecting diagnostic information from the implanted sensor means; and
external data processing means for automatically receiving and calibrating diagnostic information from the implanted communication means.
